(19) United States
(12) Patent Application Publication
(10) Pub. No.: US 2021/0393570 A1
(43) Pub. Date: Dec. 23, 2021

(54) THERAPEUTICAL COMPOSITIONS FOR USE IN THE TREATMENT OF NON-MALIGNANT CONDITIONS ASSOCIATED WITH PHOSPHATIDYLINOSITOL-3-KINASE ACTIVATION: OVERGROWTH SPECTRUM, CUTANEOUS CAPILLARY MALFORMATIONS AND SEBORRHEIC KERATOSES

(71) Applicants: UNIVERSITÉ DE BOURGOGNE, DIJON CEDEX (FR); CENTRE HOSPITALIER UNIVERSITAIRE (CHU), DIJON CEDEX (FR); INSTITUT NATIONAL DE LA SANTÉ ET DE LA RECHERCHE MÉDICALE (INSERM), Paris (FR)

(72) Inventors: Gaëtan JEGO, Quetigny (FR); Laurence JEGO, Quetigny (FR); Laurence OLIVIER-FAIVRE, Asnieres Les Dijon (FR); Carmen GARRIDO, Talant (FR); Pierre VABRES, Dijon (FR)

(21) Appl. No.: 16/759,770

(22) PCT Filed: Oct. 31, 2017
(86) PCT No.: PCT/IB2017/001491

§ 371 Date: Apr. 28, 2020

(51) Int. Cl. A61K 31/343

(52) U.S. Cl. CPC A61K 31/343

(57) ABSTRACT

A method of treatment of PIK3CA Related Overgrowth Spectrum, cutaneous capillary malformations, and seborrheic kératoses by administration, to a patient in need, of a pharmaceutical composition including an inhibitor of heat-shock factor 1 (HSF1), wherein the inhibitor of heat-shock factor 1 is selected among one or more of Triptolide, Minnelide, Kribb11, Quercetin, QC-12, Quercetin derivatives, KNK437, Stresgenin B, Emunin, NZ28, Cantharidin, Rocaglamide A, Rohinitib, Rohinitib-Cantharidin hybrids ligands, Arctigenin, and pharmaceutically acceptable derivatives, salts and solvates thereof.

### TECHNICAL FIELD

The present invention generally relates to pharmaceutical compositions for the treatment of diseases associated with phosphatidylinositol-3-kinase activation.

### BACKGROUND ART

Phosphatidylinositol-3-kinase (PI3K) activation results in various conditions in humans. Segmental overgrowth describes non-malignant hypertrophy affecting only some parts of the body, with normal growth elsewhere. In contrast with cancer, segmental overgrowth lacks cell immortality and does not result in local tissue invasion or distant metastases. Postzygotic activating mutations in the phosphatidylinositol-3-kinase (PI3KCA) gene are a major cause of segmental overgrowth. Mutations in the PIK3CA gene, which encodes the PI3K p110α catalytic subunit, are found in hot spots such as p.His1047Arg or p.His1047Leu. All overgrowth syndromes characterized by PIK3CA-activated mutation have been designated as PIK3CA Related Overgrowth Spectrum (PROS).

PIK3CA (phosphatidylinositol-3-kinase, catalytic, alpha) Related Overgrowth Spectrum (PROS) is highly variable depending on the tissue affected, and spans a wide variety of disorders or syndromes which have often had different clinical names given to them in the past. Examples of these include:
CLOVES (Congenital lipomatous overgrowth, vascular malformations and epidermal naevi, scoliosis and skeletal deformities) (ICD-10: Q873)
HHML (Hemihyperplasia-multiple lipomatosis syndrome)
Fibroadipose hyperplasia
Macrodactyly (ICD-10: Q740-Q742)
Macrodystrophica lipomatosa (ICD-10: E882)
Infiltrating facial lipomatosis (ICD-10: E882)
Klippel-Trenaunay Syndrome (KTS, ICD-10: Q872)
Megalencephaly or hemimegalencephaly syndromes ((ICD-10: Q873): Macrocephaly-capillary malformation (MOM), Megalencephaly, polymicrogyria, and hydrocephalus syndrome (MPPH), Megalencephaly-capillary malformation-polymicrogyria syndrome (MCAP), Macrocephaly-cutis marmorata telangiectasia congenita (M-CMTC).

In addition with segmental overgrowth, phosphatidylinositol-3-kinase activation may also result in localized cutaneous overgrowth or malformations:
seborrheic keratosis (ICD-10: L82 according to the International Classification of Diseases), a benign epidermal overgrowth
cutaneous capillary or capillaro-lymphatic malformation (ICD-10: 1781)
cutaneous capillary or capillaro-lymphatic malformation with overgrowth (ICD-10: Q828).

Segmental overgrowth syndromes may thus involve various tissues, resulting in subcutaneous, lipomatous, muscular, visceral, skeletal, cerebral, vascular and cutaneous hyperplasia.

Congenital PIK3CA-related conditions are rare diseases with a reduced market, with an estimated number of less than 10 000 patients in France (about 200 have been ascertained by the present Inventors) with one of the forms of the disease. In contrast, cutaneous epidermal hyperplasia due to PIK3CA activation known as seborrheic keratosis is extremely common in adults and its prevalence increases with age, with almost every individual affected after the age of 60. Cutaneous capillary malformations are not uncommon

At present the main treatment for overgrowth is surgical excision of tissue, or surgery to reduce growth. Thanks to the identification of signaling pathways and the proteins involved, it is conceivable that new targeted therapies can be tested in patients. As no curative treatments are hitherto available to treat segmental overgrowth syndromes, there is a need to develop such treatments. Likewise, treatment of seborrheic keratoses mainly consists of local ablative methods (cryotherapy, electrodessication, curettage, laser).

### Technical Problem

It is an object of the present invention to provide a pharmaceutical composition for use in the treatment of PIK3CA Related Overgrowth Spectrum, cutaneous capillary malformations, and seborrheic keratoses.

### General Description of the Invention

In order to overcome the above-mentioned problem, the present invention proposes a pharmaceutical composition for use in the treatment of PIK3CA Related Overgrowth Spectrum, cutaneous capillary malformations, and seborrheic keratoses comprising an inhibitor of heat-shock factor 1 (HSF1), wherein the inhibitor of heat-shock factor 1 is selected among one or more of Triptolide; Minnelide; Kribb11; Quercetin; QC-12; Quercetin derivatives, preferably Fisetin, Naringenin, Kaempferol and Baicalein; KNK437; Stresgenin B; Emunin; NZ28; Cantharidin; Rocaglamide A; Rohinitib; Rohinitib-Cantharidin hybrids ligands, preferably RC1 to RC20; Arctigenin; and pharmaceutically acceptable derivatives, salts and solvates thereof.

The invention is based on the surprising finding that a composition able to inhibit heat-shock factor 1 is useful in treating PIK3CA Related Overgrowth Spectrum. Actually, heat shock transcription factor 1 (HSF1) is the major stress-responsive transcription factor. It is constitutively expressed in all tissues, but its activation is only observed in conditions of cellular stress. HSF1 is activated by trimerization and hyperphosphorylation. Originally, it was described to bind to heat shock elements in the promoter region of genes involved in the response to heat shock and to positively regulate their expression. This induction results in the accumulation of molecular chaperones with anti-aggregation properties, called heat shock proteins (HSPs), such as HSP70 with, as a final outcome, cell protection. HSF1 is also involved in development and cellular differentiation, by regulating non-heat shock genes including a wide set of genes related to various processes such as apoptosis, RNA splicing, and ubiquitination.

It appears that patients with somatic activating mutations in the phosphatidylinositol-3-kinase gene (PIK3CA) show various segmental overgrowth syndromes. The present Inventors have investigated the involvement of HSF1 in the development of these diseases.

The Inventors show that HSF1 is over-activated in patients in vivo and in their in vitro skin fibroblasts. Furthermore, HSF1 functional inhibition leads to a reduction in cell cycle and cell growth of those cells.

According to the present invention, the inhibitor of heat-shock factor 1 is selected e.g. among one or more of Triptolide; Minnelide; Kribb11; Quercetin; QC-12; Quercetin derivatives: such as preferably Fisetin, Naringenin, Kaempferol and Baicalein; KNK437; Stresgenin B; Emunin; NZ28; Cantharidin; Rocaglamide A; Rohinitib; Rohinitib-Cantharidin hybrids ligands, such as preferably RC1 to RC20; Arctigenin, as well as pharmaceutically acceptable derivatives, salts and solvates thereof.

Triptolide is a diterpenoid epoxide (Formula I) which is endogenously produced by the thunder god vine, Tripterygium wilfordii.

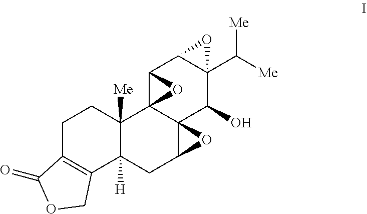 [SMILES: CC(C)[C@]12O[C@H]1[C@@H]1O[C@]13[C@]1(O[C@H]1C[C@H]1C4=C(CC[C@@]13C)C(=O)OC4)[C@@H]2O]

Minnelide (Formula II) is O-phosphonooxymethyltriptolide, e.g. as disodium salt.

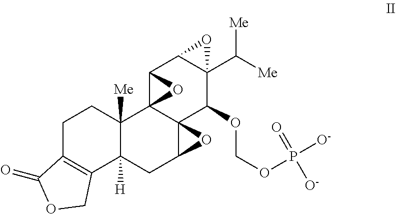 [SMILES: CC(C)[C@]12O[C@H]1[C@@H]1O[C@]13[C@]1(O[C@H]1C[C@H]1C4=C(CC[C@@]13C)C(=O)OC4)[C@@H]2OCOP(=O)([O-])[O-]]

Kribb11 can be represented by Formula III.

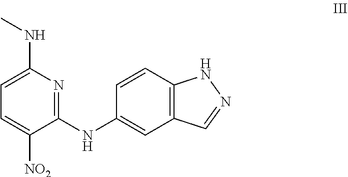 [SMILES: CNc1ccc([N+](=O)[O-])c(Nc2ccc3[nH]ncc3c2)n1]

Quercetin can be represented by Formula IV.

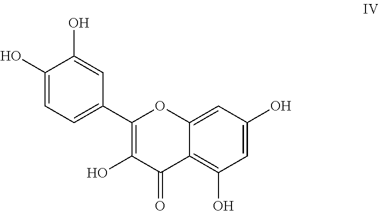 [SMILES: O=c1c(O)c(-c2ccc(O)c(O)c2)oc2cc(O)cc(O)c12]

QC-12, a soluble pro-drug metabolized into quercetin, can be represented by Formula V.

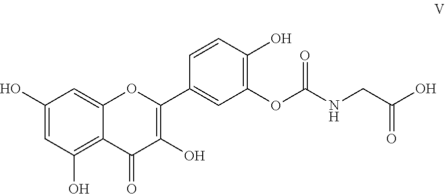 [SMILES: O=C(O)CNC(=O)Oc1cc(-c2oc3cc(O)cc(O)c3c(=O)c2O)ccc1O]

Fisetin, a quercetin derivative, can be represented by Formula VI.

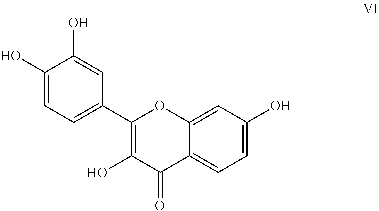 [SMILES: O=c1c(O)c(-c2ccc(O)c(O)c2)oc2cc(O)ccc12]

Naringenin, a quercetin derivative, can be represented by Formula VII.

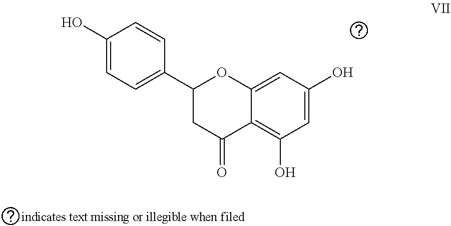 [SMILES: O=C1CC(c2ccc(O)cc2)Oc2cc(O)cc(O)c21]

Kaempferol, a quercetin derivative, can be represented by Formula VIII.

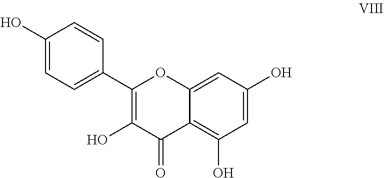 [SMILES: O=c1c(O)c(-c2ccc(O)cc2)oc2cc(O)cc(O)c12]

Baicalein, a quercetin derivative, can be represented by Formula IX.

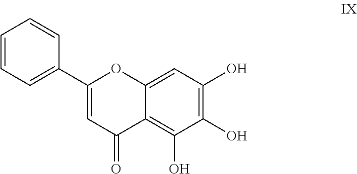 [SMILES: O=c1cc(-c2ccccc2)oc2cc(O)c(O)c(O)c12]

KNK437 can be represented by Formula X.

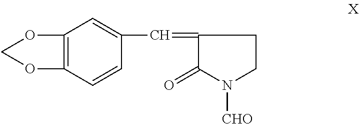 [SMILES: O=CN1CCC(=Cc2ccc3c(c2)OCO3)C1=O]

Stresgenin B can be represented by Formula XI.

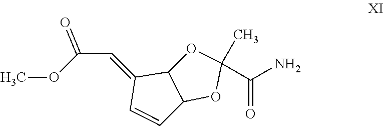 [SMILES: COC(=O)/C=C1\C=CC2OC(C)(C(N)=O)OC12]

Emunin can be represented by Formula XII.

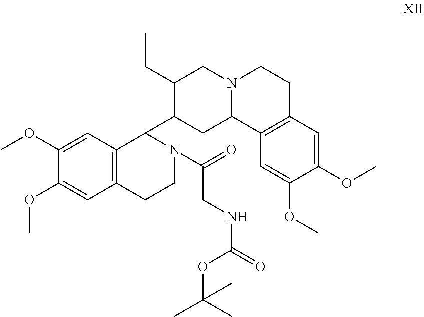

NZ28 can be represented by Formula XIII.

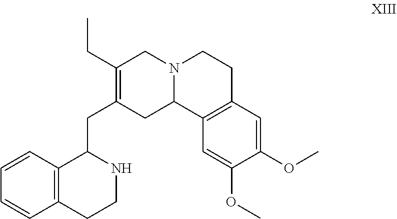 [SMILES: CCC1=C(CC2NCCc3ccccc32)CC2c3cc(OC)c(OC)cc3CCN2C1]

Cantharidin can be represented by Formula XIV.

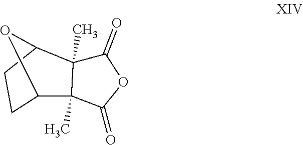

Rocaglamide can be represented by Formula XV.

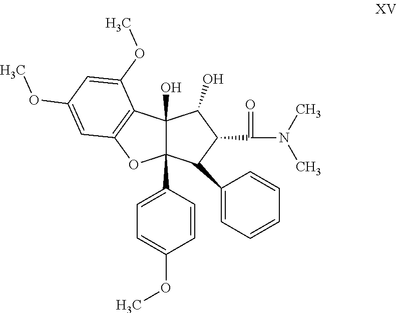

Rohinitib can be represented by Formula XVI.

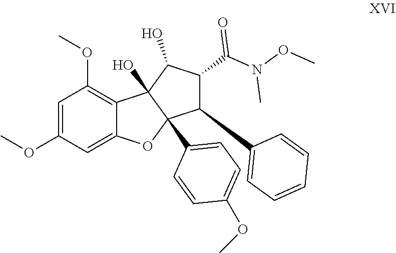

Rohinitib and Cantharidin hybrids ligands RC1 to RC20 can be represented by Formulae XVII RC1 to RC20.

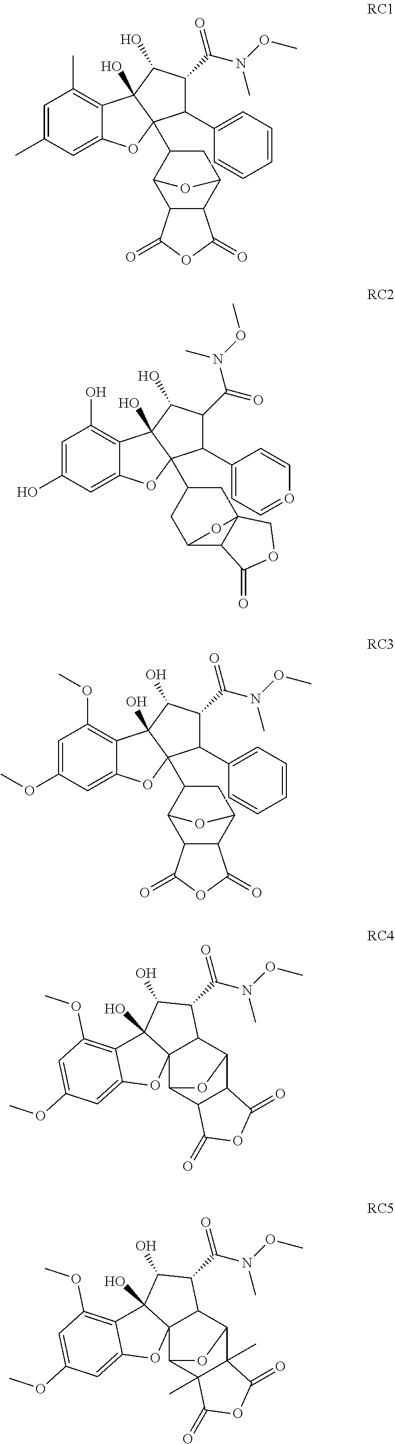

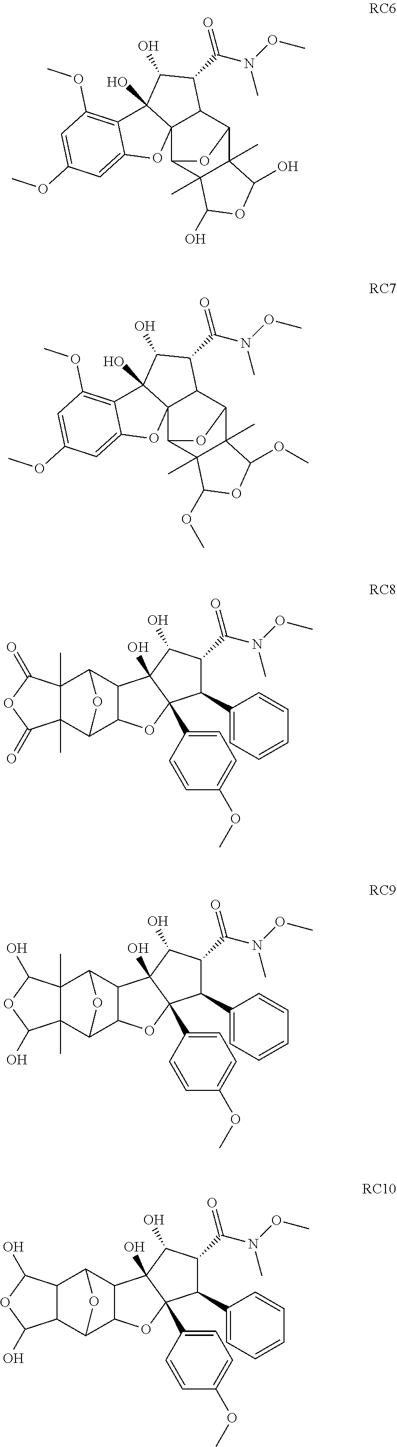

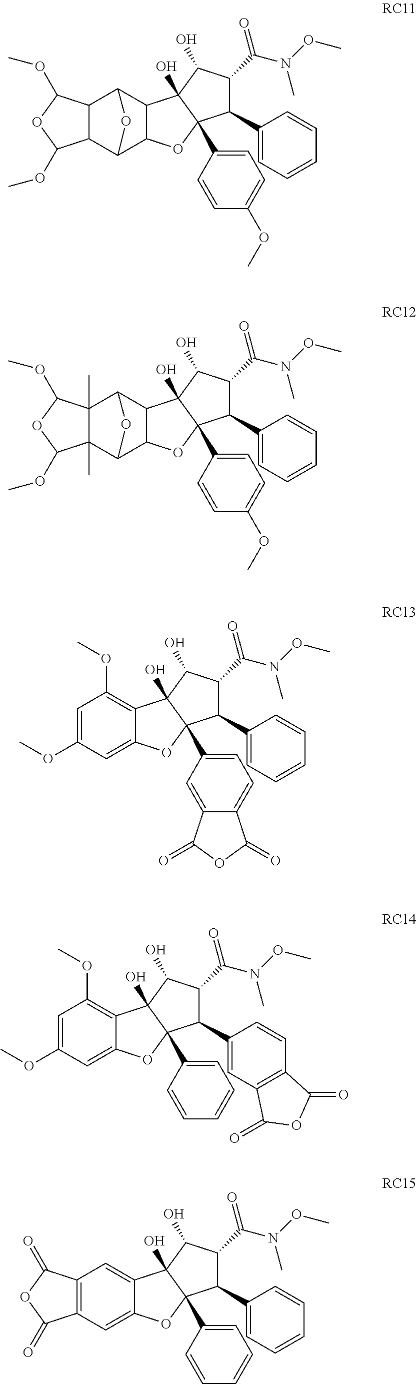

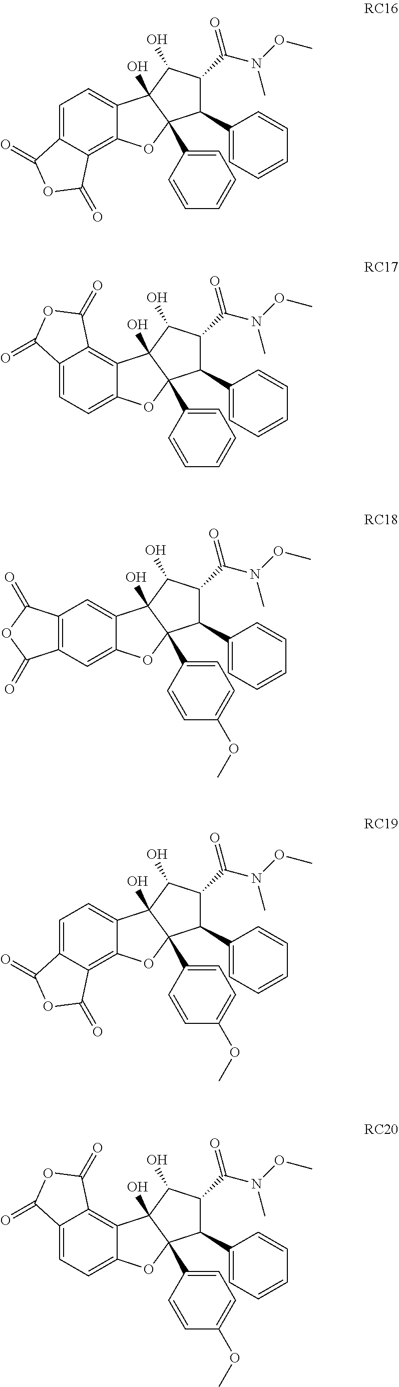 [SMILES: CON(C)C(=O)[C@H]1[C@@H](O)[C@@]2(O)c3ccc4c(c3O[C@@]2(c2ccccc2)[C@@H]1c1ccccc1)C(=O)OC4=O ; CON(C)C(=O)[C@H]1[C@@H](O)[C@@]2(O)c3c(ccc4c3C(=O)OC4=O)O[C@@]2(c2ccccc2)[C@@H]1c1ccccc1 ; COc1ccc([C@@]23Oc4cc5c(cc4[C@]2(O)[C@H](O)[C@H](C(=O)N(C)OC)[C@H]3c2ccccc2)C(=O)OC5=O)cc1 ; COc1ccc([C@@]23Oc4c(ccc5c4C(=O)OC5=O)[C@]2(O)[C@H](O)[C@H](C(=O)N(C)OC)[C@H]3c2ccccc2)cc1 ; COc1ccc([C@@]23Oc4ccc5c(c4[C@]2(O)[C@H](O)[C@H](C(=O)N(C)OC)[C@H]3c2ccccc2)C(=O)OC5=O)cc1]

Arctigenin can be represented by the Formula XVIII.

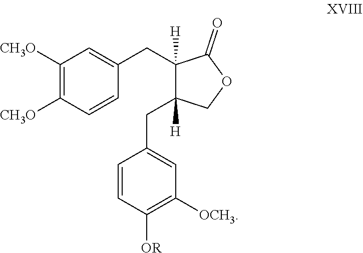

Pharmaceutical compositions according to the invention are useful in the treatment of PIK3CA Related Overgrowth Spectrum, cutaneous capillary malformations, and seborrheic keratoses. As used herein the “PIK3CA Related Overgrowth Spectrum” refers to patients with segmental hypertrophy or other cerebral, skeletal, vascular, adipocytic, cutaneous manifestations linked to a mosaic mutation of the gene PIK3CA.

As used herein the term “treatment” refers to the inhibition of the evolution, particularly the regression, preferentially the disappearance of the pathology and/or the symptoms.

As used herein the term “pharmaceutically acceptable” refers to molecular entities and compositions that do not produce undue adverse, allergic or other untoward reaction when administered to a mammal, especially a human, as appropriate.

As used herein the term “derivatives” refers to all derivatives of the compound having an efficiency equivalent to or greater than the compound itself. By “equivalent efficiency” is meant that the compound derivative will have an efficiency between 50 and 100%, preferably between 80 and 100% of that of the compound. Therefore, by “equivalent or greater efficiency” is meant that the compound derivative will have an efficiency of at least 50%, preferentially at least 80%, of that of the compound.

As used herein the term “salts” or “pharmaceutically acceptable salts” of a compound, refers to a salt which is pharmaceutically acceptable as defined herein and which possesses the desired pharmacological activity of the parent compound. The pharmaceutically acceptable salts include:

(1) pharmaceutically acceptable acid salts formed with pharmaceutically acceptable inorganic acids such as hydrochloric acid, hydrobromic acid, sulfuric acid, nitric acid, phosphoric acid and like; or formed with pharmaceutically acceptable organic acids such as acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphthoic acid, 2-hydroxy-ethanesulfonic acid, lactic acid, maleic acid, malic acid, mandelic acid, methane-sulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like, and

(2) pharmaceutically acceptable base salts formed when an acid proton present in the parent compound is either replaced by an metallic ion, for example an alkali metal ion, an alkaline earth metal ion or an aluminum ion; is coordinated with an organic base pharmaceutically acceptable such as diethanolamine, ethanolamine, N-methylglucamine, triethanolamine, tromethamine and the like; or with an inorganic base pharmaceutically acceptable such as aluminum hydroxide, calcium hydroxide, potassium hydroxide, sodium carbonate, sodium hydroxide and similar. It may be a sodium salt when the compound has an acid function.

As used herein the term “solvates” refers to a compound formed by solvation, for example as a combination of solvent molecules with molecules or ions of a solute. Well known solvent molecules include water, alcohols and other polar organic solvents. Alcohols include methanol, ethanol, n-propanol, isopropanol, n-butanol, isobutanol, and t-butanol. Alcohols also include polymerized alcohols such as polyalkylene glycols (e.g. polyethylene glycol, polypropylene glycol). The best-known and preferred solvent is typically water, and solvate compounds formed by solvation with water are termed hydrates.

The present document further describes a method of treatment of PIK3CA Related Overgrowth Spectrum, cutaneous capillary malformations, and seborrheic keratoses by administration to a patient in need of a pharmaceutical composition according to the invention.

The present invention further relates to the use of a pharmaceutical composition according to the invention in the manufacture of a medicament for the treatment of PIK3CA Related Overgrowth Spectrum, cutaneous capillary malformations, and seborrheic keratoses.

In a particular embodiment of the invention, the pharmaceutically acceptable derivatives of Triptolide are selected among triptonide, tripdiolide, triptolidenol, 16-hydroxytriptolide, triptriolide, 12-epitriptriolide, 14-epi-triptolide, tripchlorolide and the derivative PG-490-88.

In some embodiments, the pharmaceutical composition further comprises at least one pharmaceutically acceptable carrier or excipient. The desired or necessary active compound of the present invention (e.g. Triptolide, Minnelide or Kribb11, Quercetin, QC-12, Quercetin derivatives: Fisetin, Naringenin, Kaempferol and Baicalein, KNK437, Stresgenin B, Emunin, NZ28, Cantharidin, Rocaglamide A, Rohinitib, Rohinitib-Cantharidin hybrids ligands: RC1 to RC20, Arctigenin) may be combined with pharmaceutically acceptable excipients or carriers.

The expression “a pharmaceutically acceptable carrier or excipient” refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The pharmaceutical compositions of the present invention can be administered in a unit administration form, as a mixture of the active ingredient(s) with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

### BRIEF DESCRIPTION OF THE DRAWINGS

Preferred embodiments of the invention will now be described, by way of example, with reference to the accompanying drawings in which:

FIG. 1 Immunoblotting showing an increase in phosphorylation of HSF1 and AKT in patients' fibroblasts compared to controls.

FIG. 2 (A) Treatment with insulin induces the phosphorylation of AKT and HSF1. (B) Inhibition of AKT phosphorylation by LY294002 (LY) and Rapamycin (Rapa) inhibits phosphorylation of HSF1. (C) Rapamycin and LY294002 inhibit the nuclear and cytoplasmic localization of P-HSF1. (D) Quantitative PCR analysis of the expression of 74 genes belonging to a cancer-related gene signature in patient A fibroblasts after treatment with LY294002. A code with different patterns is used to illustrate the intensity of the increase (gradient of grey) or decrease (shading pattern) of gene expression compared to untreated cells;

FIG. 3 Cell viability measured by XTT labeling after 48 h of treatment by 10 μM LY294002 or by increasing doses of Triptolide (A), by 4 ng/ml Rapamycin (B) or increasing doses of Kribb11. For each experiment: control 1 (white bar), control 2 (hatched bar), patient B (grey bar), patient A (black bar). n=3. O.D.=optical density. (C) Number of cells after 48 h treatment for patient A. (D) Cell proliferation measured by incorporation of BrdU. O.D.=optical density. (E) Fibroblasts apoptosis of patients and healthy donors measured by a cell death detection ELISA kit, after 24 h treatment with 100 ng/ml rapamycin, 10 μM LY294002, 3 μM Kribb11 or 10 nM Triptolide. 1 μM of staurosporine was used as an inducer of apoptosis and as positive control of the experiment, n=3. O.D.=optical density. *p<0.05, **p<0.01, ***p<0.005.

Further details and advantages of the present invention will be apparent from the following detailed description of several not limiting embodiments with reference to the attached drawings.

### DESCRIPTION OF PREFERRED EMBODIMENTS

Two PROS patients were studied. High-throughput sequencing on cutaneous fibroblasts of patients allowed the identification of a mutation PIK3CA p.His1047Arg in 50% of the alleles of patient A (100% of mutated cells), as well as a rarer mutation p.Cys378Tyr in 30% of the alleles of patient B (60% of mutated cells). In both patients, the mutated cells had a higher rate of proliferation than the control cells and possess a stronger phosphorylation of AKT and p70S6K.

The Active Form of HSF1 is Present in Mutated PIK3CA Tissues and in Skins Fibroblasts of Patients with PROS.

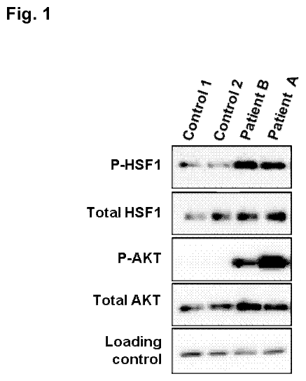

Epidermal tissues of mutated PIK3CA patients were labeled for presence of P-AKT and P-HSF1 by immunohistochemistry. The Inventors observed a high expression of P-HSF1 only in cells with high expression of P-AKT, suggesting an association between the PI3K/AKT pathway and activation of HSF1 in these patients. To deepen this correlation, the Inventors used fibroblasts of patients A and B as well as 2 non-mutated donors, for which they compared the levels of P-HSF1 (FIG. 1). In agreement with their mutational status, a strong phosphorylation of AKT was observed only in patients A and B. Despite the detection of the phosphorylated form in control fibroblasts, the phosphorylated form is stronger in the cells of patients. Since the culture of cells of patient A consists solely of mutated cells, they were exclusively used for the study. As a transcription factor, active HSF1 must be located in the nucleus. A nuclear localization of HSF1 has been observed by immunofluorescence in the cells of patient A, whereas the nuclear localization is low in cells control.

The Activation of HSF1 is Reduced by Inhibitors of the PI3K/AKT Pathway

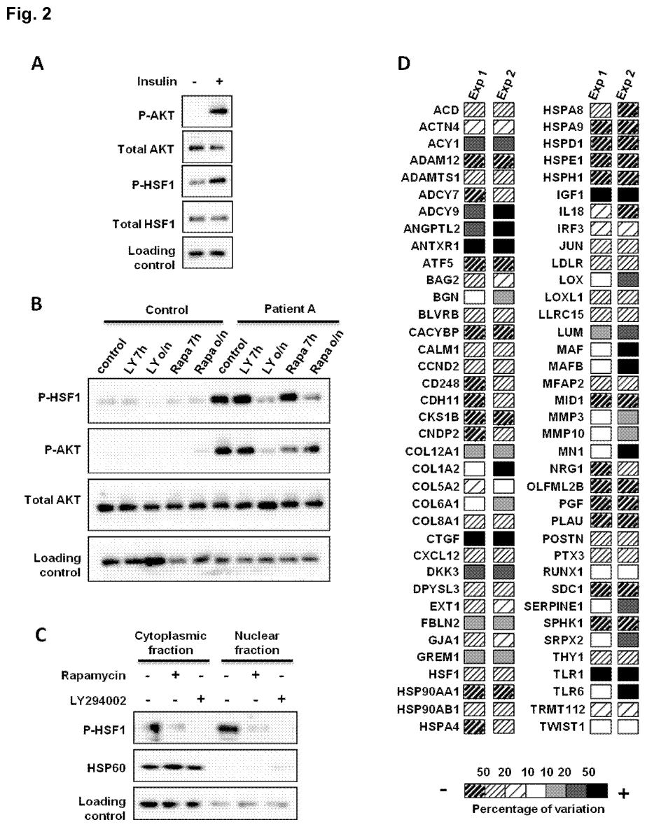

The Inventors have further investigated whether the PI3K/AKT/mTOR pathway is directly responsible for the activation of HSF1 in the fibroblasts of patients. The induction of PI3K/AKT activating pathway by adding insulin to normal fibroblasts is well known. Similarly, the Inventors observed an induction of the phosphorylation of HSF1 under these conditions (FIG. 2A), suggesting that it is not strictly dependent on the mutation but rather it is the consequence of hyperactivation of the PI3K/AKT pathway. In addition, the inhibition of the activity of PI3K or mTOR, by the specific inhibitors, LY294002 and Rapamycin respectively, reduces the phosphorylation of HSF1 in the control and in the patient cells (FIG. 2B). Moreover, P-HSF1 is no longer localized in the nucleus of cells after treatment (FIG. 2C), which suggests that the expression of the HSF1-dependent genes is inhibited accordingly. To test this hypothesis, the expression of 74 known genes to be regulated by HSF1 have been determined after treatment with the PI3K inhibitor (FIG. 2D). In two independent experiments, the expression of 47 genes is inhibited while the expression of 11 genes is increased. The results are variable for 12 other genes (increase and decrease). Interestingly in this signature, the CCND2 gene coding for cyclin D2 is significantly decreased after treatment with LY294002 (FIG. 2D). Thus, a large part of a HSF1-dependent gene signature can be inhibited by the blocking of PI3K and thus confirms the link between these two pathways in the syndrome of segmental hypertrophy PIK3CA dependent.

The Targeting of HSF1 with Specific Inhibitors Reduces the Proliferation of Cells of Patients with PROS.

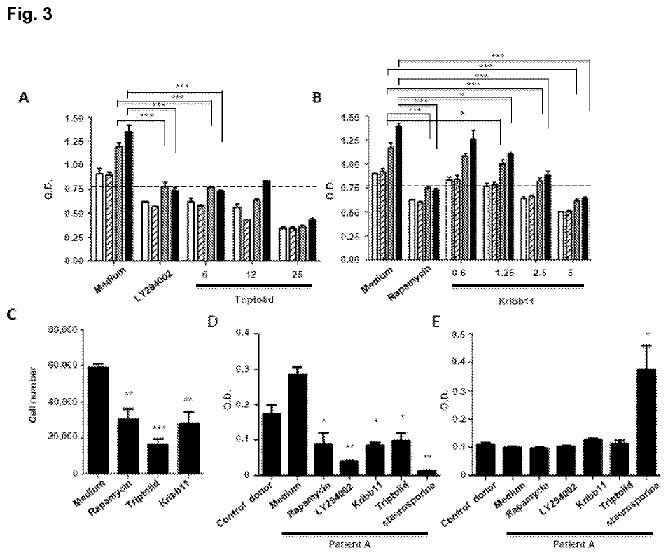

The incubation of fibroblasts from patients with specific HSF1 inhibitors, e.g. Triptolide and Kribb11, induces a reduction in cell growth and in the number of total cells as effectively as inhibitors of PI3K or mTOR (FIGS. 3A, 3B and 3C). The increase in the % of cells entering the S phase which is characteristic of cells of hypertrophy syndrome cells is completely blocked by the Kribb11 (FIG. 3D). This inhibition of proliferation is not associated with an increase in cell mortality as shown in FIG. 3E.

1. A method of treatment of PIK3CA Related Overgrowth Spectrum, cutaneous capillary malformations, and seborrheic keratoses by administration, to a patient in need, of a pharmaceutical composition comprising an inhibitor of heat-shock factor 1 (HSF1), wherein the inhibitor of heat-shock factor 1 is selected among one or more of Triptolide; Minnelide; Kribb11; Quercetin; QC-12; Quercetin derivatives; KNK437; Stresgenin B; Emunin; NZ28; Cantharidin; Rocaglamide A; Rohinitib; Rohinitib-Cantharidin hybrids ligands; Arctigenin; and pharmaceutically acceptable derivatives, salts and solvates thereof.

2. The method according to claim 1, wherein the pharmaceutically acceptable derivatives of Triptolide are selected among triptonide, tripdiolide, triptolidenol, 16-hydroxytriptolide, triptriolide, 12-epitriptriolide, 14-epi-triptolide, tripchlorolide and the derivative PG-490-88 or combinations thereof.

3. The method according to claim 1, wherein the Quercetin derivatives are selected among Fisetin, Naringenin, Kaempferol and Baicalein or combinations thereof.

4. The method according to claim 1, wherein the Rohinitib-Cantharidin hybrid ligands are selected among hybrid ligand compounds RC1 to RC20: 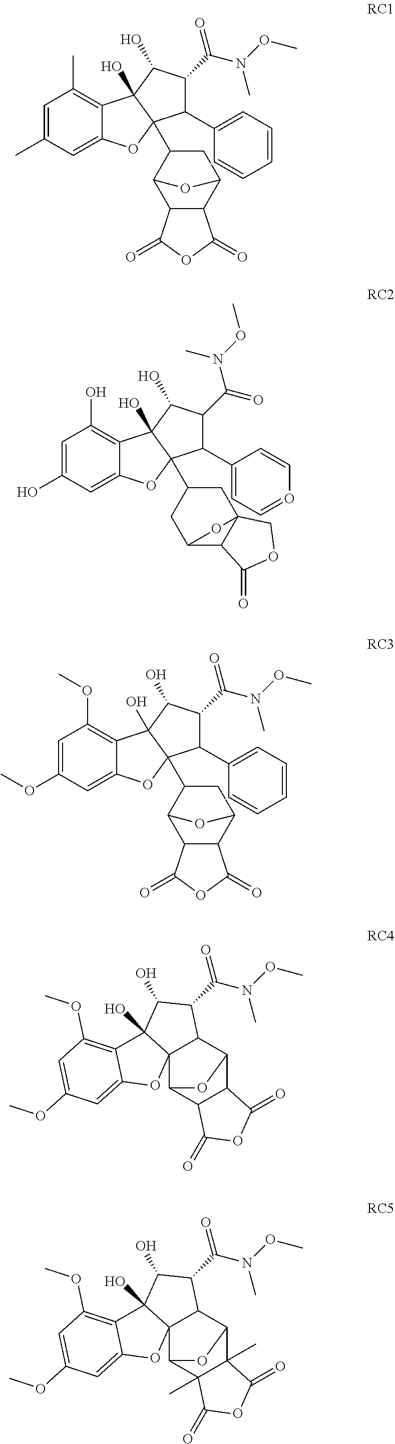 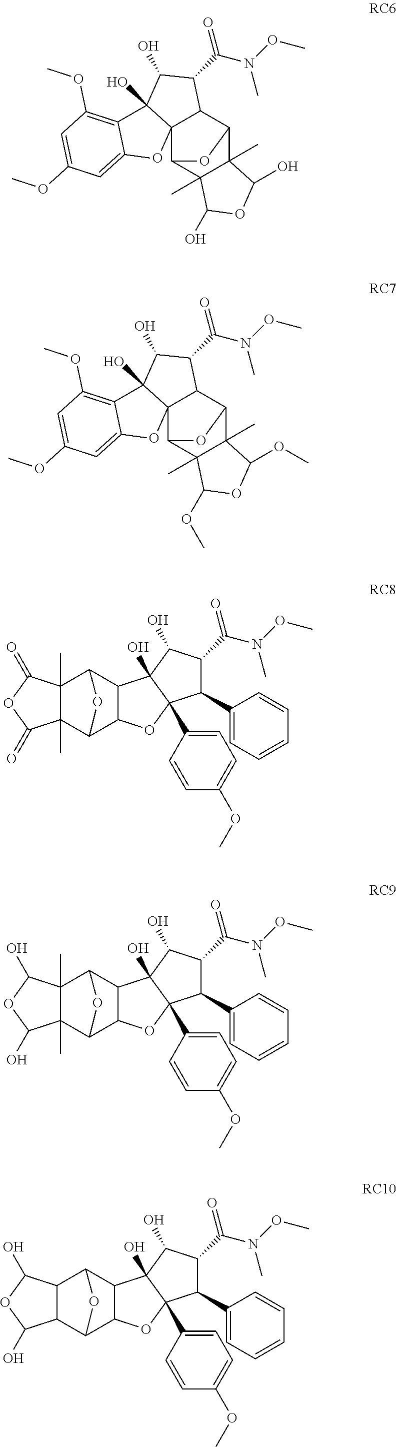 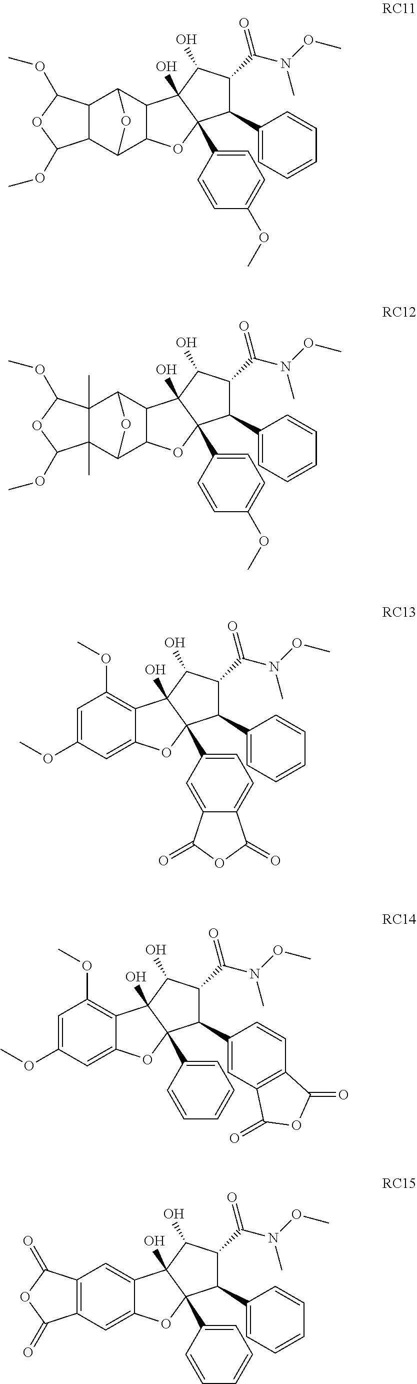 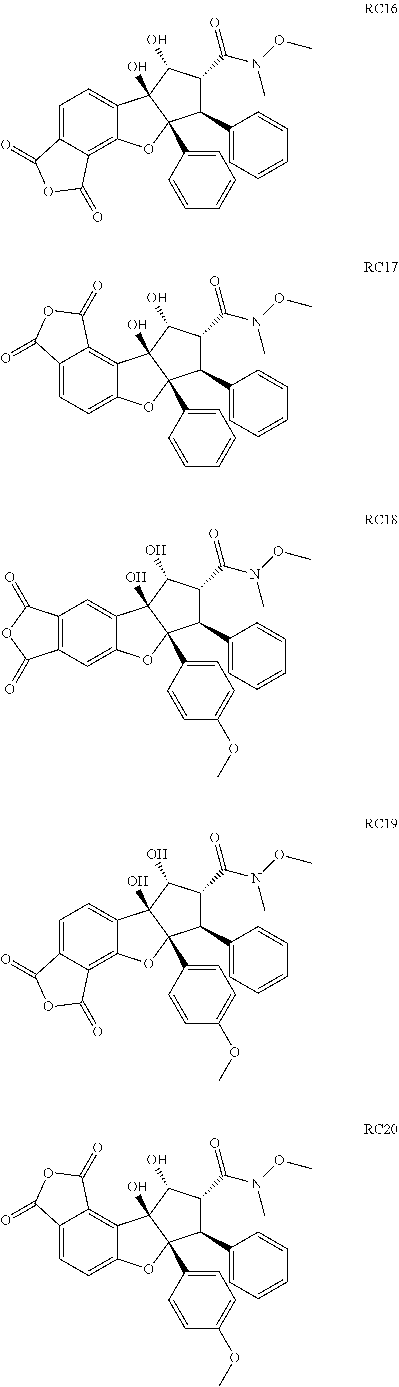 [SMILES: CON(C)C(=O)[C@H]1[C@@H](O)[C@@]2(O)c3ccc4c(c3O[C@@]2(c2ccccc2)[C@@H]1c1ccccc1)C(=O)OC4=O ; CON(C)C(=O)[C@H]1[C@@H](O)[C@@]2(O)c3c(ccc4c3C(=O)OC4=O)O[C@@]2(c2ccccc2)[C@@H]1c1ccccc1 ; COc1ccc([C@@]23Oc4cc5c(cc4[C@]2(O)[C@H](O)[C@H](C(=O)N(C)OC)[C@H]3c2ccccc2)C(=O)OC5=O)cc1 ; COc1ccc([C@@]23Oc4c(ccc5c4C(=O)OC5=O)[C@]2(O)[C@H](O)[C@H](C(=O)N(C)OC)[C@H]3c2ccccc2)cc1 ; COc1ccc([C@@]23Oc4ccc5c(c4[C@]2(O)[C@H](O)[C@H](C(=O)N(C)OC)[C@H]3c2ccccc2)C(=O)OC5=O)cc1]
or combinations thereof.

5. The method according to claim 1, further comprising at least one pharmaceutically acceptable carrier or excipient.

6. The method according to claim 1, wherein said composition is in a solid, semisolid, gel, suspension, or emulsion form.

7. The method according to claim 1, wherein said composition is in a liquid form.

8. The method according to claim 1, wherein said pharmaceutical composition is administered orally, intravenously or topically.
